(19) United States
(12) Patent Application Publication
(10) Pub. No.: US 2022/0061819 A1
(43) Pub. Date: Mar. 3, 2022

(54) Ultrasound Image Reading Method and System Thereof

(71) Applicants: China Medical University, Taichung City 404 (TW); China Medical University Hospital, Taichung City 404 (TW)

(72) Inventors: Jui Fang, Taipei City (TW); Yi-Wen Chen, Taichung City (TW); Hsin-Yuan Fang, Taichung City (TW)

(21) Appl. No.: 17/367,757

(22) Filed: Jul. 6, 2021

(30) Foreign Application Priority Data
Sep. 2, 2020 (TW) 109129969

(51) Int. Cl. A61B 8/08

(52) U.S. Cl. CPC A61B 8/5269

(57) ABSTRACT

An ultrasound image reading method has steps as follows: (1) reading an ultrasound image; (2) labeling artifacts in the ultrasound image; (3) identifying features of the artifacts and transforming the features into parameters, and using the parameters to determine an artifacts combination; and (4) using the artifacts combination to look up a corresponding score of disease.

### TECHNICAL FIELD

The present disclosure relates to a method and system which read and utilize an ultrasound image to judge a tissue lesion in a body.

### RELATED ART

Ultrasound has been the most basic and important detection tool in medicine and disease treatment. Through ultrasound imaging, technicians and physicians can understand the health status of the internal tissues and organs of the human body. This is one of the fastest and most effective medical testing methods. The existing ultrasound operation practice is to scan the affected part of the patient with an ultrasound probe by an operating technician/physician, and an ultrasound image formed based on the reflected ultrasound signal received by the ultrasound probe to achieve the purpose of medical judgment.

The current ultrasound image often generates some image signals called artifacts in the ultrasound image due to various reasons of in-body and imaging. These artifacts were considered as meaningless error signals in the early days. However, many studies have proved that, some artifacts are related to the internal symptoms of the patients.

The existing interpretation of artifacts is usually subjective, for example, manually calculating the number of artifacts to determine the relationship between artifacts and internal disease signs, and this approach is difficult to objectively quantify and compare.

### SUMMARY

In order to solve the technical problems of the existing ultrasound image judgment technology that the existing interpretation method for certain meaningful artifacts of the ultrasound image is too subjective and without quantitative judgment, which can only be explained through experience, the present invention proposes an ultrasound image reading method for disease judgment. The ultrasound image reading method comprises steps of:
reading an ultrasound image;
labeling artifacts in the ultrasound image;
identifying features of the artifacts, obtaining artifacts parameters and determining an artifacts combination according to the artifacts parameters; and
finding a score of disease according to the artifacts combination.

According to the above features, the ultrasound image reading method further comprising a step of analyzing intensity distribution of scatter signals in the ultrasound image, wherein the scatter signals in the ultrasound image is described by a Homodyned-K model, and then a parameter μ is obtained, the parameter μ is an effective scatter number is proportional to an actual scatter number per resolution cell.

According to the above features, the artifacts parameters comprises an attenuation slope α, a peak distance d, a regression correlation coefficient R, wherein the attenuation slope α is a slope of a peak connection in the time-domain signal of the artifacts, the peak distance d is a distance between two adjacent peaks in the time-domain signal of the artifacts, and the regression correlation coefficient R represents a difference between the peak connection and a fitting curve.

As can be seen from the foregoing descriptions, the present disclosure proposes a quantitative method for analyzing and defining ultrasound artifacts. Through this method, the type of artifacts in the ultrasound image can be analyzed, and the corresponding lesions that may occur in human tissue can be determined, so as to achieve the purpose of auxiliary diagnosis.

### BRIEF DESCRIPTIONS OF DRAWINGS

FIG. 1 is a flow chart of an ultrasound image reading method according to an embodiment of the present disclosure.

FIG. 2 is a schematic diagram of an ultrasound image.

FIG. 3A through FIG. 3D are schematic diagrams showing analysis to scatter signal distributions in the ultrasound images.

FIG. 3E is a schematic diagram showing correspondence between the scatter signal distribution and severity of illness.

FIG. 3F is a schematic diagram showing a probability density function.

FIG. 4 is a schematic diagram showing analysis to a time-domain signal of the ultrasound artifacts.

FIG. 5 is a schematic diagram showing recognition of parameters and features of the ultrasound artifacts.

FIG. 6 is a schematic diagram of an ultrasound image according to another embodiment.

FIG. 7 is a schematic diagram of analysis according to another embodiment.

### DETAILS OF DEMONSTRATED EMBODIMENT

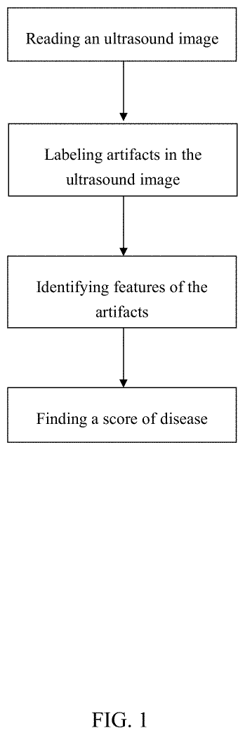

Refer to FIG. 1, which illustrates steps of an ultrasound image reading method of the present disclosure, and the steps to be executed comprise step 1 through step 4.

At step 1, an ultrasound image is read.

The ultrasound image reading system obtains the ultrasound image, and can be obtained directly by an ultrasound detection device or by a detection database.

At step 2, an artifacts in the ultrasound image is labeled.

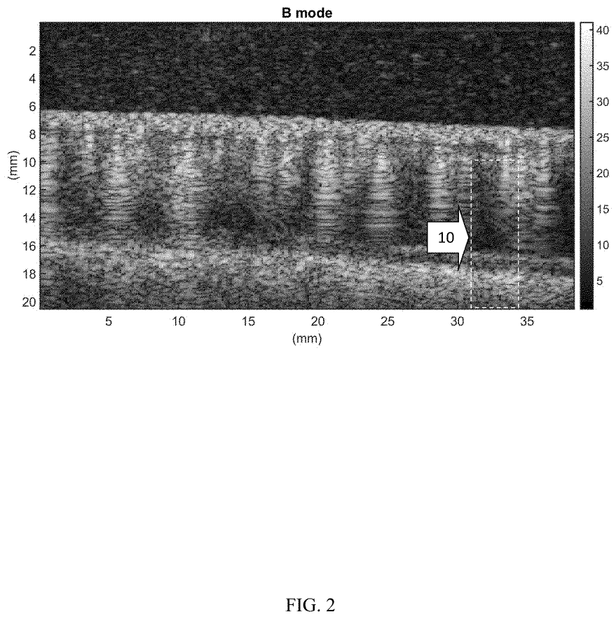

Referring to FIG. 2, and after the ultrasound image reading system obtains the ultrasound image, the artifacts 10 (B-line) is found in the ultrasound image. The causes of the artifacts 10 can be divided into two models, and they are a multiple reflections model and a scattering model which generated by the ultrasound in the human body. The two models are illustrated as follows.

In the normal detection process, a probe of the ultrasound detection device generates an ultrasound signal to penetrate into the human body. Because the compositions of the tissues or organs in the human body are different, the ultrasound penetrates the different compositions with different acoustic impedances to generate reflections. In this way, the probe reads the reflected ultrasound to form the ultrasound image. The inspector can judge whether there is abnormal tissue or disease in the body through the ultrasound image. When the transmission of the ultrasound in the body does not meet the imaging hypothesis of the ultrasound detection device, the aforementioned artifacts 10 may be generated.

Taking the ultrasound detection of the lungs as an example, interstitial lung disease may have problems such as changes in interstitial thickness and density (pulmonary edema, pneumonitis, and pulmonary fibrosis), which means that the artifacts 10 may appear in the ultrasound image. The interstitial thickness and/or density changes, so that the multiple reflections of the ultrasound appears between two or more material interfaces with different acoustic impedances, and such multiple reflections do not meet the preset imaging hypothesis of the ultrasonic detection device. Therefore, the artifacts are generated in the ultrasound image, wherein the artifacts signal intensity due to multiple reflections decreases with the increase in the number of reflections. As shown in FIG. 2, the depth direction signal of the artifacts 10 gradually attenuates and can be observed.

Regarding the artifacts 10 caused by scattering, when the size of the tissues or structures in the body is small relative to the wavelength of the incident ultrasound, the artifacts 10 is caused by the ultrasound scattering. Taking the lung as an example, the tissues or structures include, for example, alveoli with smaller sizes, and interstitium with local disease.

At step 3, the features of the artifacts are identified.

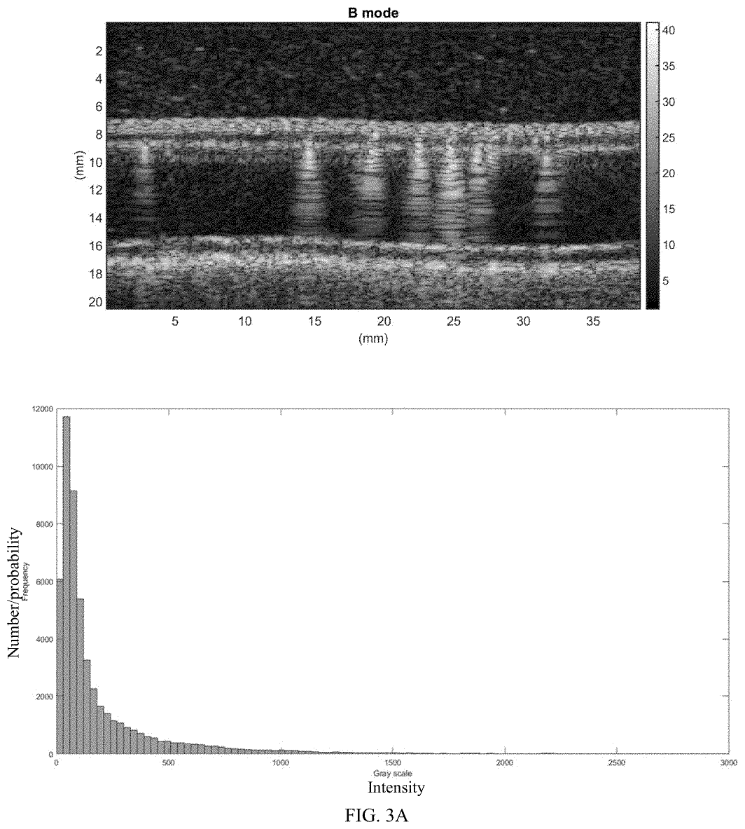

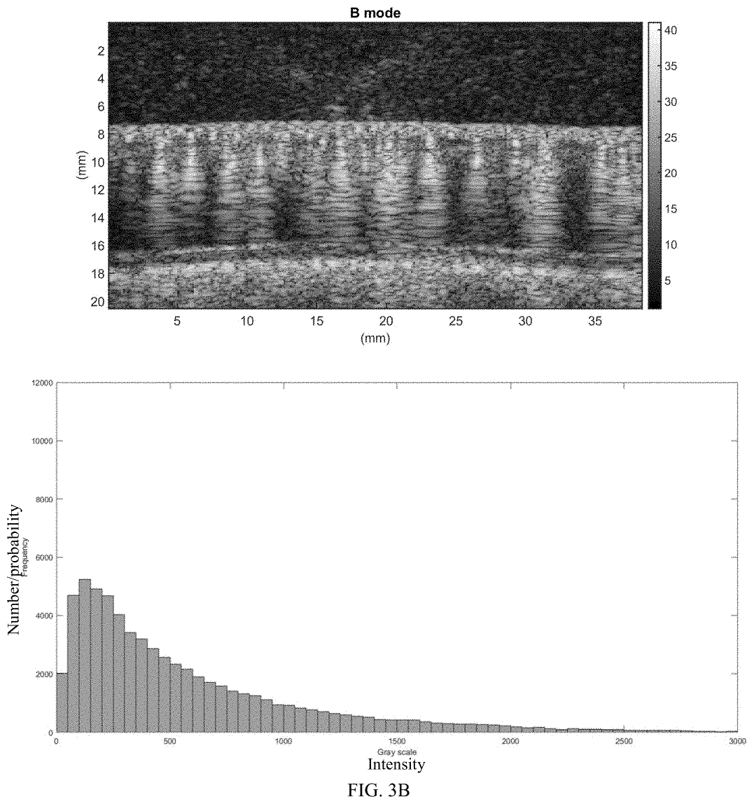

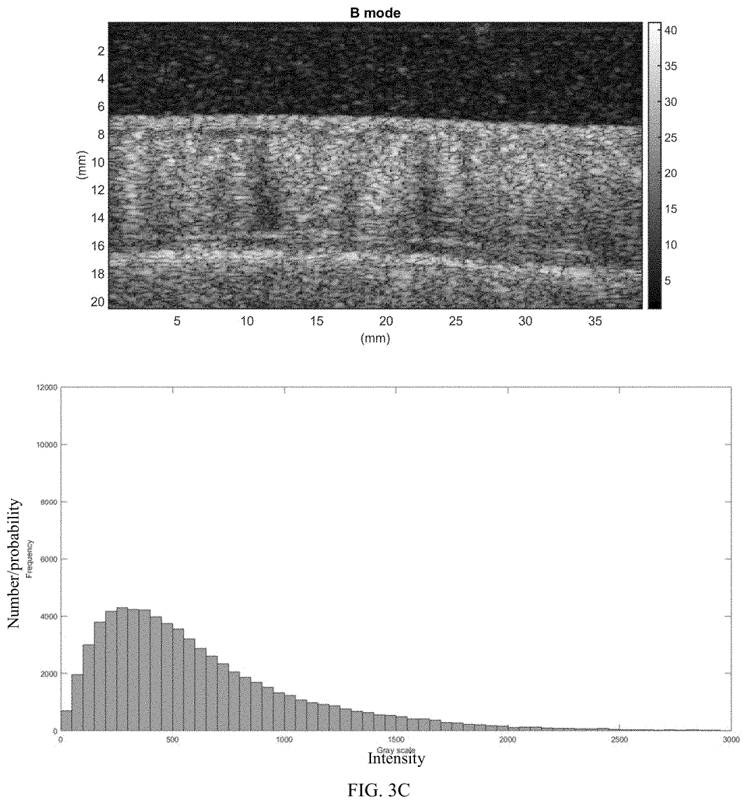

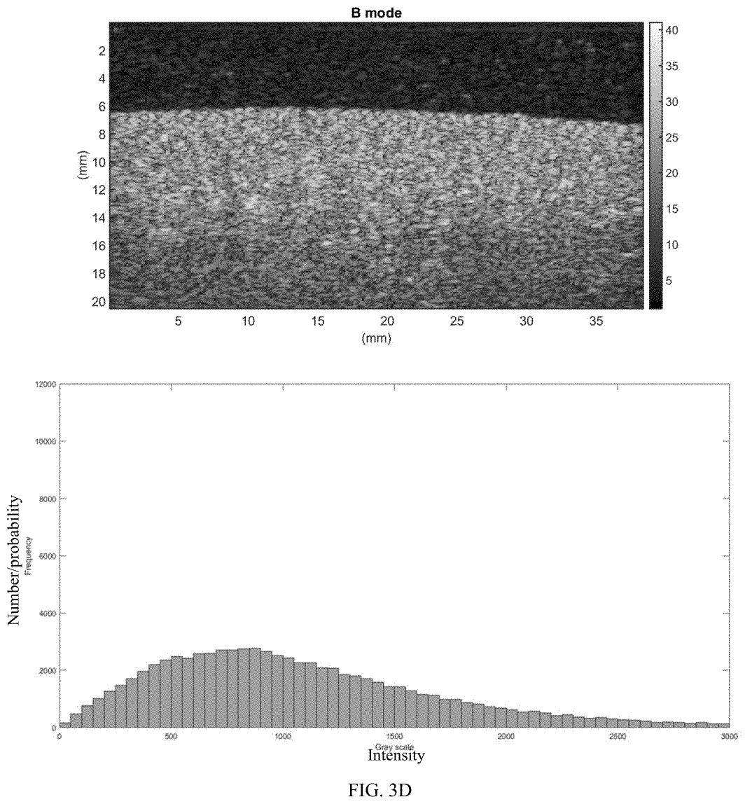

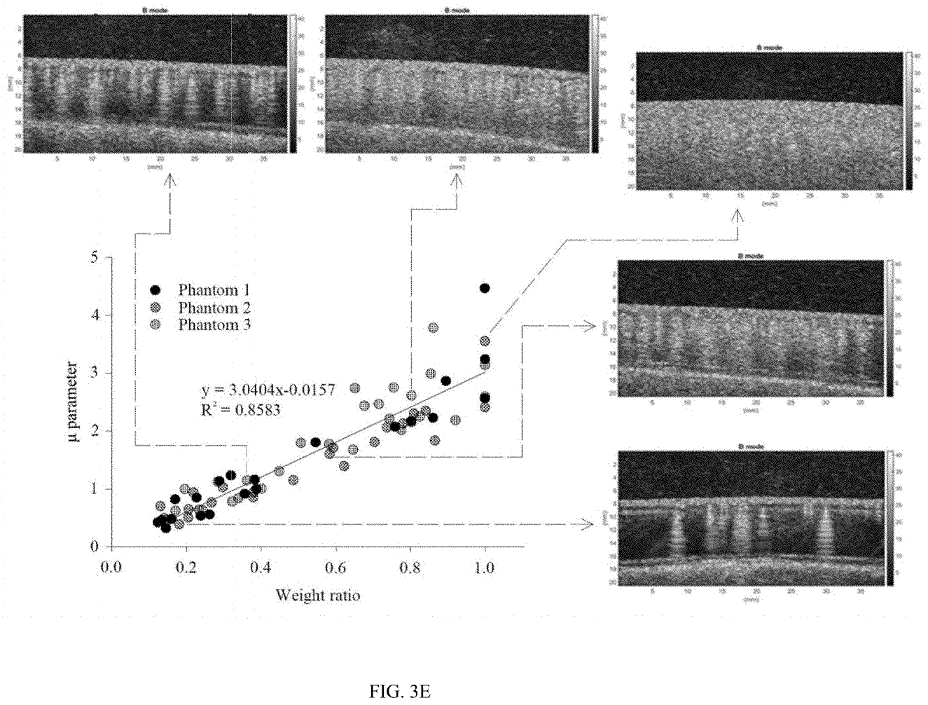

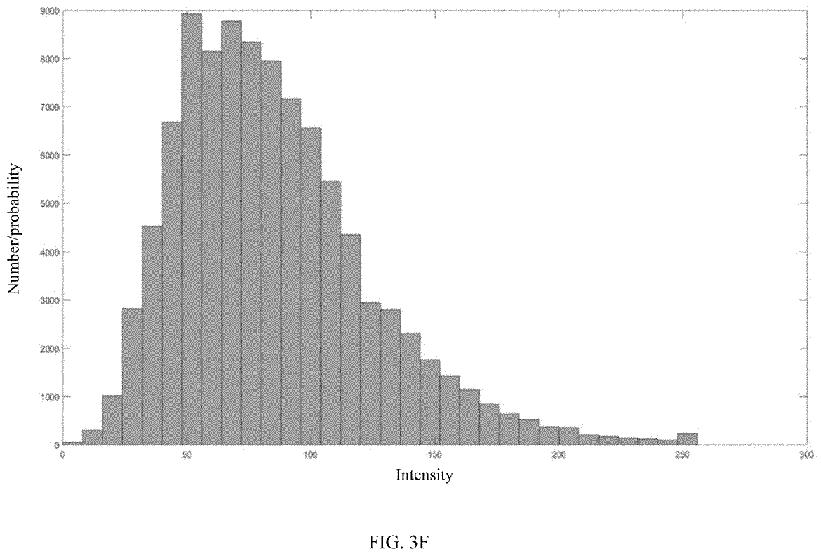

Refer to FIG. 3A through FIG. 3D, and the ultrasound image reading system performs a scattering signal analysis and calculation on the ultrasound image to obtain the intensity distribution result of the ultrasound image. The horizontal axis is the signal intensity, and the vertical axis is the number of the intensity of the signal or occurrence probability of the intensity of the signal. The intensity distribution status can correspond to the ultrasound image.

In order to quantize the aforementioned scattering signals, the ultrasound image can be analyzed through Homodyned-K distribution model to obtain an effective scatter number parameter (i.e. μ parameter). The effective scatter number parameter is proportional to the actual number of scatter number per resolution cell. As shown in FIG. 3E, it can be seen that the effective scatter number μ can present the different types of the artifacts 10. When the ultrasound image presents more scatter types patterns of the artifact 10, the parameter of the effective scatter number μ calculated by the Homodyned-K model is relatively large. Therefore, using this proportional relationship, the artifact 10 in the ultrasound image can be used to estimate possible pathological phenomena.

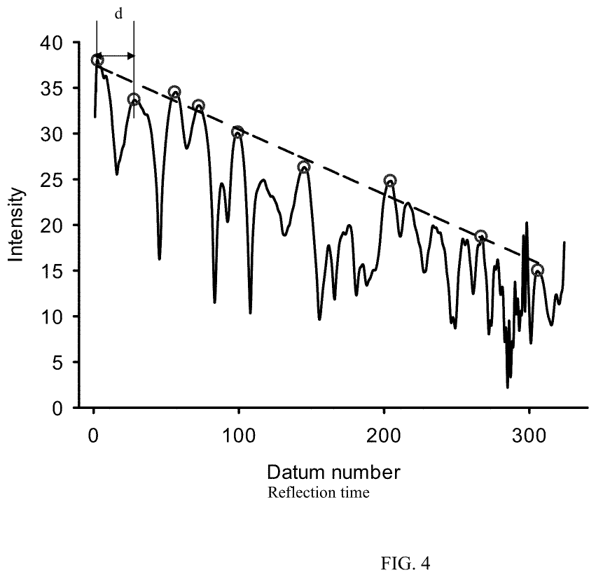

Several methods of analyzing ultrasound signals from the viewpoint of scattering are based on the probability density function, which is to calculate the number of occurrences of each intensity in the image and then divide by the total number of all intensities, so as to obtain the probability of each intensity. In FIG. 4F,the probability density function is graphed, wherein the horizontal axis is the intensity, and the vertical axis is the probability of occurrences.

In addition to the number of the effective scatter (μ) parameter of the aforementioned Homodyned-K model, the probability density function can also be described by a Shannon entropy model, and the calculation is expressed by equation (1):

H_{c}≡−∫ₐ^{b}ω(y)log₂[ω(y)]dy (1)

wherein ω(y) is the probability density function of each image intensity, a and b are the minimum and maximum image intensity.

Shannon entropy model is a measurement model of uncertainty. When the random signal generated in the ultrasound image is large, the Shannon entropy (H_{c}) is larger, in other words, the probability of various image intensities appearing is more average, the Shannon entropy is greater. When the Shannon entropy (H_{c}) after ultrasound image analysis is smaller, it can present that the more B-line artifacts in the lung is, the more serious the disease is.

In addition, it can also be represented by a skewness, which is used to measure the asymmetry of the probability density function. When the skewness is negative, it means that most of the values of the probability density function are on the right side of the average value. Conversely, when the skewness is positive, it means that most of the probability density function values are on the left side of the average value. When the skewness is zero, it means that the values of the probability density function are evenly distributed on both sides of the average, but it is not necessarily symmetrical. When the skewness after ultrasound image analysis is greater, the number of B-line artifacts is smaller and the disease is milder.

To analyze the ultrasound signal from the perspective of the reflection, an A-mode signal analysis (time-domain signal) can be used. The A-mode signal analysis is the result of each longitudinal signal in the ultrasound image expressing the brightness of an image with a numerical intensity. Refer to FIG. 4,and in order to get the intensity of one of the artifactsl0 at different reflection times (from Datum number), the aforementioned reflection time can be converted to the distance. In the condition of artifacts 10 caused by lung lesions in this embodiment, each peak represents a reflection signal of the ultrasound in an interstitium of the lesion, wherein an attenuation slope α0 of a peak connection in the artifact 10 are related to the acoustic resistance of the medium material . Taking abnormal lung lesions as an example, the attenuation slope α may represent edema, fibrosis, and congestion in the lungs. Therefore, by analyzing the attenuation slope α, the type of disease can be inferred. The attenuation slope α is the feature of the A-mode signal, which can be used as a basis for judging different diseases. The tissue abnormalities caused by different diseases have different acoustic impedance, and the reflection caused by it will make the attenuation of the A-mode signal is unique.

A peak distance d between the two peaks of the artifact 10 represents the thickness change of the reflective material of the artifact 10, which may be the thickness of the interstitium in the lungs.

The peak distance d presents the severity of a certain type of disease. The aforementioned peak distance d is the local maximum value of the A-mode signal, which can represent the peak. The distance between the two peaks can represent the time difference between the two reflections. The larger the reflection time difference is, the longer the distance between the two reflection interfaces is, the larger the lesion and the more serious the disease are.

When the ultrasound is output from the probe to the human body, there may be multiple sources of reflection or scattering due to the distribution of the lesions in the body, so that the formed artifact 10 is a superposition of multiple reflection source signals, so the connection of the peak in FIG. 4 is usually not a theoretical curve. By calculating a regression correlation coefficient R between a curve of the actual peak connection and a fitting result of the peak connection, it can be used to estimate the condition and severity of the disease contained in the human body. Taking pulmonary edema as an example, if the degree of pulmonary edema is not high and the range is small, the ultrasound image may only contain one line of the artifacts 10, and the peak line of the artifacts 10 is closer to a fitting curve. Conversely, if the pulmonary edema in the lungs becomes more serious, the A-mode signals generated by multiple lesions will be superimposed at the same position and there will be constructive and destructive interference, so that the attenuation slope α of the peaks of the artifacts 10 will be relatively small, the regression correlation coefficient R is relatively small, there may be a variety of the peak distances d, and the peak distances d are relatively high. The aforementioned regression correlation coefficient R is the difference between the actual attenuation of the A-mode signal and the theoretical attenuation. The smaller the correlation coefficient R is, the more the lesions are, the more severe the disease is.

It can be seen that by analyzing and recognizing the artifact 10, the artifacts parameters of the artifact 10 can be obtained including attenuation slope α, peak distance d, and regression correlation coefficient R.

The reason for the generation of the artifacts 10 will be different due to the different morphology of tissue lesions in the body. In the pulmonary lesion example of this embodiment, due to factors such as the ratio of water to air in the lung, lesion area, etiology and so on, the appearance of the artifacts 10 generated by multiple reflections and scattering of the ultrasound is different.

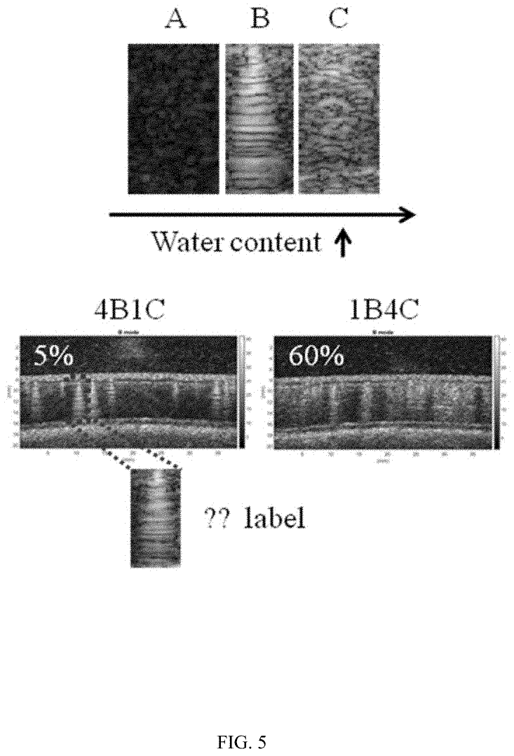

Taking pulmonary edemaas an example, please refer to FIG. 5. In this embodiment, as the severity of pulmonary edemais different, three different types of artifacts 10 can be defined as A, B, and C, respectively. The degree of pulmonary edemais C>B>A.

The bottom left image of FIG. 5 has 4 pieces of the artifacts 10 of type B and one piece of the artifacts 10 of type C, representing that one of the artifacts combination of the ultrasound image of the bottom left image is 4B1C, and the bottom right of FIG. 5 is one ultrasound image having a corresponding artifacts combination of 1B4C.

In each ultrasound image, the number of artifacts 10 existing in different regions can be defined by the aforementioned image recognition method, and the artifacts parameters and the artifacts combination of each artifact 10 can be defined at the same time. In this way, the values can be integrated, and the expression types of the artifacts 10 in the ultrasound image can be quantitatively calculated.

At step 4, the score of disease is found.

After completing the aforementioned qualitative calculation of artifacts 10 and the artifacts combination, a database can be used to determine a score of disease corresponding to a different artifacts combination, wherein the database stores the artifacts combination that correspond to different types of disease and severity. For example, the artifacts combination in the lower left and right lower pictures of FIG. 5 corresponds to the case of pulmonary edema. Since there are 4 type B artifacts with medium severity of pulmonary edema and 1 type C artifacts with high severity of pulmonary edema in the ultrasound image in the lower left picture of FIG. 5. It means that although there is pulmonary edema in the corresponding area of the lungs of the observed human body, it is not serious. With direct observation of pathology and anatomy, the database can summarize the pulmonary edema scores of different types of artifacts. For example, the score of B type artifacts 10 in the lungs is B, corresponding to the weight X, and the score of type C artifacts 10 in the lungs is C, and the corresponding weight is Y. The degree of pulmonary edema corresponding to the ultrasound image in the lower left picture of FIG. 5 is 4*B*X+1*C*Y=5%, and the degree of the pulmonary edema corresponding to the ultrasound image in the bottom right picture of FIG. 5 is 1*B*X+4*C*Y=60%.The weight can be defined based on the results of clinical pathological research. The database can store a comparison table, or the parameters are used to define the corresponding weight data that may be generated by different tissue locations and disease types.

If the observed tissue regions are different, similar or identical artifacts types may be generated. The database can store the artifacts types of different body regions and their corresponding pathological severity according to clinical research. In other words, according to different tissue locations, different types of disease, and severity, the different types of artifacts 10 are produced corresponding to the record. By recognizing the type and quantity of artifacts, preliminary statistics can be made on the type and severity of disease that may occur in the corresponding human tissue area of the captured ultrasound image. For example, in addition to the aforementioned lung diseases, such as pulmonary edema, pulmonary fibrosis, pneumonitis, etc., other examples are illustrated as follows.

### EXAMPLE 1

### Foreign Body

For example, due to accidents such as car accidents, explosions, or falls, and objects that enter the body through the orifices of the body such as swallowing or inhalation, according to their different properties from body tissues, they will leave artifacts in the ultrasound image, and the artifacts of different types and degrees can be used to judge the locations, types and sizes of foreign objects, which can be used as a basis for treatment planning.

### EXAMPLE 2

### Free Air in Abdominal or Pelvic Cavity

Normally, only a small amount of air bubbles in the digestive tract in the abdominal cavity or pelvic cavity will produce ultrasonic artifacts, so a large amount or abnormal location of gas can be distinguished by artifacts. The appearance of a large amount of gas in the intestine is often related to bowel necrosis. If gas is found around an abscess, it is likely to be a gas-forming pyogenic abscess, and its mortality rate is higher than other abscesses. Gas in the gallbladder or kidney may be caused by emphysematous inflammation, and gas in unspecified places may be caused by gastrointestinal tract perforation. These diseases can all produce different degrees of artifacts as described in this embodiment.

### EXAMPLE 3

### Gallbladder Disease

The gallbladder is usually bile, without ultrasound reflection, and it is black on the image, but abnormal tissue proliferation or deposition will produce ultrasound artifacts in places where there is no signal for reasons like lung interstitial diseases. Therefore, artifacts can be used to judge the severity of the disease, and can even be used as a basis for benign and malignant abnormal tissues. For example, Gallbladder adenomyomatosis is a proliferative disease of epidermal cells and smooth muscles on the gallbladder mucosa; cholesterol gallstone or cholesterolosis of the gallbladder is a disease in which cholesterol crystals are deposited in the gallbladder or on the gallbladder wall.

### EXAMPLE 4

### Thyroid Nodule

Thyroid nodule is a local mass in the thyroid, which is caused by the abnormal proliferation of local thyroid cells, among which benign nodules often have punctate echogenic foci with artifacts and free distribution, so they can be used to judge the benign and malignant nodules.

Further, in addition to the judgment method of image recognition, the numerical analysis results, such as, the attenuation slope α (identification of disease type), the peak distance d, the regression correlation coefficient R, the effective scatter number (μ) parameter, etc., in the aforementioned artifacts parameters can be used for judgment accompanying with the score result obtained corresponding to the clinical pathological data.

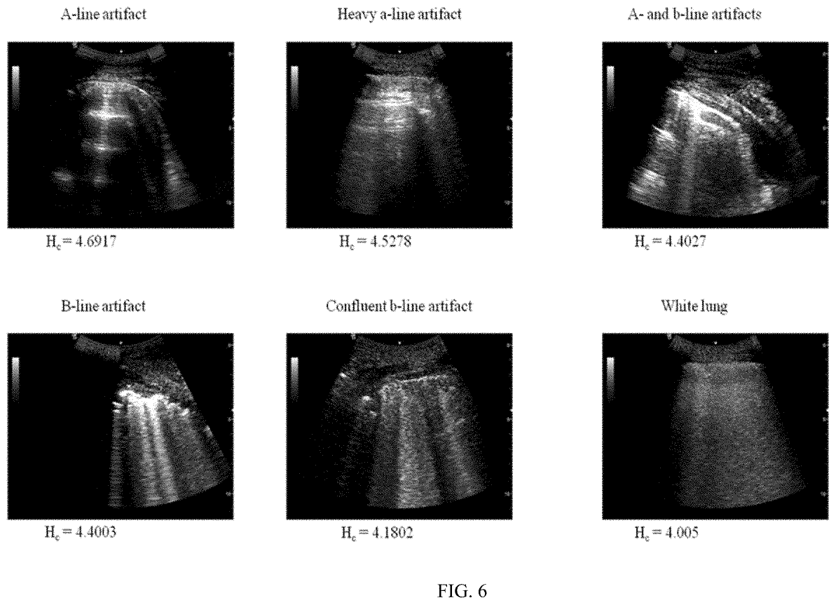

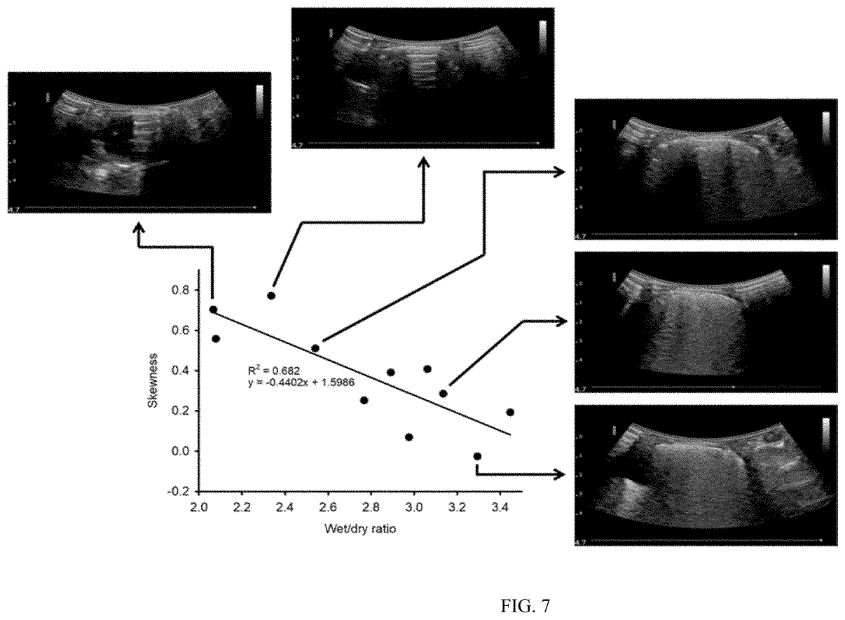

Please refer to FIGS. 6 and 7, which are the ultrasound images of the clinical pulmonary edema of the present disclosure. The name on FIG. 6 is the result of clinical subjective judgment, and the H_{c} value under FIG. 6 is the Shannon entropy of the scattering quantitative parameter. From left to right and from top to bottom in FIG. 6, the pulmonary edema is getting serious, and H_{c} decreases with the severity. FIG. 7 is a rabbit animal model based on pulmonary edema. The vertical axis in FIG. 7 is the skewness of the scattering quantitative parameter, and the horizontal axis is the severity of pulmonary edema. Different images are representative images of different degrees of pulmonary edema. From this, it can be seen that the present disclosure can correspond to the actual condition of specific symptoms through the analysis results of the disclosed ultrasound artifacts.

In the actual implementation of the foregoing embodiments, the foregoing ultrasound image reading system is preferably integrated and installed in the ultrasound detection device, or directly read/input the detection result of the ultrasonic detection device. The ultrasound image reading system includes at least a computer host, a database connected to the computer host, and an input/output interface, wherein the database is used to store at least the ultrasound image, the artifacts combination, a clinicopathological result, and relation data of artifacts features. With the aforementioned analysis modes, the computer host includes at least one multiple reflection analysis module, one scattering analysis module, and one image recognition module. The operator can select the multiple reflection analysis module and/or the scattering analysis module through the input interface (or the computer host obtains the ultrasound image) to drive the multiple reflection analysis module, the scattering analysis module, and the image recognition module to finish the multiple reflection analysis and parameters calculation, scattering analysis and parameters calculation and artifacts type recognition. Then, the artifacts combination in the ultrasound image can be correspondingly summarized, and finally compared with the pathological data of the database to determine the score of disease.

According the foregoing descriptions, the present disclosure proposes a quantitative method for analyzing and defining ultrasound artifacts. Through this method, the type of artifacts in the ultrasound image can be analyzed, and the corresponding lesions that may occur in human tissue can be determined, so as to achieve the purpose of auxiliary diagnosis.

What is claimed is:

1. An ultrasound image reading method, comprising steps of:
reading an ultrasound image;
identifying features of the artifacts by obtaining artifacts parameters according to an intensity distribution and a time-domain signal, and determining an artifacts combination according to the artifacts parameters; and
finding a score of disease according to the artifacts combination.

2. The ultrasound image reading method of claim 1, wherein the artifacts combination is classified by counting numbers of different types of the artifacts in the ultrasound image.

3. The ultrasound image reading method of claim 1, further comprising a step of analyzing intensity distribution of the ultrasound image, wherein a probability density function is used to describe, so as to obtain a parameter based on the probability density function, and the parameter is used to describe an in-body scattering status.

4. The ultrasound image reading method of claim 3, wherein the artifacts parameters comprise an attenuation slope, a peak distance, and a regression correlation coefficient, wherein the attenuation slope is a slope of a peak connection in the time-domain signal of the artifacts, the peak distance is a distance between two adjacent peaks in the time-domain signal of the artifacts, and the regression correlation coefficient is a correlation of the peak connection and a fitting curve.

5. The ultrasound image reading method of claim 2, further comprising a step of analyzing intensity distribution of the ultrasound image, wherein a probability density function is used to describe, so as to obtain a parameter based on the probability density function, and the parameter is used to describe an in-body scattering status.

6. The ultrasound image reading method of claim 5, wherein the artifacts parameters comprise an attenuation slope, a peak distance, and a regression correlation coefficient, wherein the attenuation slope is a slope of a peak connection in the time-domain signal of the artifacts, the peak distance is a distance between two adjacent peaks in the time-domain signal of the artifacts, and the regression correlation coefficient is a correlation of the peak connection and a fitting curve.

7. An ultrasound image reading system, comprising a computer host and a database connected to the computer host, wherein the database is used to store a ultrasound image, an artifacts combination, a clinicopathological result and a relation data of artifacts features; the computer host at least comprises a multiple reflection analysis module, a scattering analysis module and an image recognition module, the computer host read the ultrasound image, and respectively drives the multiple reflection analysis module, the scattering analysis module and the image recognition module to finish a multiple reflection analysis and parameters calculation, a scattering analysis and parameters calculation and an artifacts type recognition, wherein the computer host executes an ultrasound image reading method which comprises steps of:
reading an ultrasound image;
labeling artifacts in the ultrasound image;
identifying features of the artifacts by obtaining artifacts parameters according to an intensity distribution and a time-domain signal, and determining an artifacts combination according to the artifacts parameters; and
finding a score of disease according to the artifacts combination.

8. The ultrasound image reading system of claim 7, wherein the artifacts combination is classified by counting numbers of different types of the artifacts in the ultrasound image.

9. The ultrasound image reading system of claim 7, wherein the ultrasound image reading method further comprises a step of analyzing intensity distribution of the ultrasound image, wherein a probability density function is used to describe, so as to obtain a parameter based on the probability density function, and the parameter is used to describe an in-body scattering status.

10. The ultrasound image reading system of claim 9, wherein the artifacts parameters comprise an attenuation slope, a peak distance, and a regression correlation coefficient, wherein the attenuation slope is a slope of a peak connection in the time-domain signal of the artifacts, the peak distance is a distance between two adjacent peaks in the time-domain signal of the artifacts, and the regression correlation coefficient is a correlation of the peak connection and a fitting curve.

11. The ultrasound image reading system of claim 8, wherein the ultrasound image reading method further comprises a step of analyzing intensity distribution of the ultrasound image, wherein a probability density function is used to describe, so as to obtain a parameter based on the probability density function, and the parameter is used to describe an in-body scattering status.

12. The ultrasound image reading system of claim 11, wherein the artifacts parameters comprise an attenuation slope, a peak distance, and a regression correlation coefficient, wherein the attenuation slope is a slope of a peak connection in the time-domain signal of the artifacts, the peak distance is a distance between two adjacent peaks in the time-domain signal of the artifacts, and the regression correlation coefficient is a correlation of the peak connection and a fitting curve.
